(19) United States
(12) Patent Application Publication
(10) Pub. No.: US 2023/0062506 A1
(43) Pub. Date: Mar. 2, 2023

(54) METHOD AND SYSTEM FOR PANDEMIC AND CONNECTION TRACKING

(71) Applicants: Qatar University, Doha (QA); Qatar Foundation for Education, Science and Community Development, Doha (QA)

(72) Inventors: Ala Gouissem, Doha (QA); Khalid Abualsaud, Doha (QA); Elias Yaacoub, Doha (QA); Tamer Khattab, Doha (QA); Mohsen Guizani, Doha (QA)

(21) Appl. No.: 17/900,275

(22) Filed: Aug. 31, 2022

Related U.S. Application Data
(60) Provisional application No. 63/238,901, filed on Aug. 31, 2021.

(51) Int. Cl. G16H 50/80; H04W 4/02; H04W 4/029; H04W 4/021

(52) U.S. Cl. CPC G16H 50/80; H04W 4/023; H04W 4/029; H04W 4/021

(57) ABSTRACT

A system for pandemic and connection tracking is provided. The system includes a memory and one or more processors in communication with the memory, and a data collector executing on the one or more processors. The data collector is configured to collect anonymous tracking data from a wireless sensor network. The wireless sensor network is configured to communicate with users' devices using a wireless connection. The system further includes a data validator/augmentor executing on the one or more processors. The data validator/augmentor is configured to validate the collected anonymous tracking data. The system also includes a virus spread tracker executing on the one or more processors. The virus spread tracker is configured to: estimate an exposure time between each pair of users based on the collected anonymous tracking data; and determine a probability of at least one of the users being infected based on the estimated exposure time.

### PRIORITY CLAIM

The present application claims priority to and the benefit of U.S. Provisional Patent Application No. 63/238,901, filed on Aug. 31, 2021, the entirety of which is incorporated herein by reference.

### BACKGROUND

One of the methods being used to help reduce the spread of COVID-19 is to identify infected individuals so that those individuals can be quarantined and thus limit the spread of the disease. In addition to identifying infected individuals, most methods try to warn those that have come in contact with the infected so they may also quarantine. The current methods of tracing are either interviewing infected individuals directly or through contact tracing mobile applications. Both of these approaches have potential issues operating at scale or implicate privacy issues.

### SUMMARY

The present disclosure generally relates to a method and system for pandemic and connection tracking.

In light of the present disclosure, and without limiting the scope of the disclosure in any way, in an aspect of the present disclosure, which may be combined with any other aspect listed herein unless specified otherwise, a system for pandemic and connection tracking is provided. The system includes a memory and one or more processors in communication with the memory, and a data collector executing on the one or more processors. The data collector is configured to collect anonymous tracking data from a wireless sensor network. The wireless sensor network is configured to communicate with users' devices using a wireless connection. The system further includes a data validator/augmentor executing on the one or more processors. The data validator/augmentor is configured to validate the collected anonymous tracking data. The system also includes a virus spread tracker executing on the one or more processors. The virus spread tracker is configured to: estimate an exposure time between each pair of users based on the collected anonymous tracking data; and determine a probability of at least one of the users being infected based on the estimated exposure time.

In an aspect of the present disclosure, which may be combined with any other aspect listed herein unless specified otherwise, the anonymous tracking data comprises beacons transmitted from the user's devices.

In an aspect of the present disclosure, which may be combined with any other aspect listed herein unless specified otherwise, the anonymous tracking data comprises a MAC address of the user's device.

In an aspect of the present disclosure, which may be combined with any other aspect listed herein unless specified otherwise, the data validator/augmentor is further configured to: determine whether any anonymous tracking data is missing; and responsive to determining that any anonymous tracking data is missing, augment the collected anonymous tracking data.

In an aspect of the present disclosure, which may be combined with any other aspect listed herein unless specified otherwise, validating the collected anonymous tracking data comprises comparing an average distribution of the collected anonymous tracking data with a predetermined model.

In an aspect of the present disclosure, which may be combined with any other aspect listed herein unless specified otherwise, validating the collected anonymous tracking data comprises comparing an average distribution of exposure times in the collected anonymous tracking data with an average distribution of exposure times derived using a predetermined model.

In an aspect of the present disclosure, which may be combined with any other aspect listed herein unless specified otherwise, the system further comprises a plurality of wireless sensors in the wireless sensor network configured to detect the anonymous tracking data and transmit the anonymous tracking data to the data collector.

In an aspect of the present disclosure, which may be combined with any other aspect listed herein unless specified otherwise, the plurality of wireless sensors comprise a set of sensors surrounding a location of interest for wireless triangulation.

In an aspect of the present disclosure, which may be combined with any other aspect listed herein unless specified otherwise, the probability of at least one of the users being infected is determined further based on a distance level of the pair of users during the estimated exposure time.

In an aspect of the present disclosure, which may be combined with any other aspect listed herein unless specified otherwise, the distance level comprises: a high risk distance level indicating that a distance between the pair of users is lower than a first predetermined distance; a medium risk distance level indicating that the distance between the pair of users is greater than the first predetermined distance but lower than a second predetermined distance; and a low risk distance level indicating that the distance between the pair of users is greater than the second predetermined distance.

In an aspect of the present disclosure, which may be combined with any other aspect listed herein unless specified otherwise, the system further comprises a data security protector executing on the one or more processors, wherein the data security protector is configured to detect a security attack.

In an aspect of the present disclosure, which may be combined with any other aspect listed herein unless specified otherwise, detecting the security attack comprises: recording a distance between one of the users and other users among the users for a set period of time, calculating an average distribution of the distance between the one of the users and the other users; and measuring a Hellinger distance error based on the calculated average distribution.

In light of the present disclosure, and without limiting the scope of the disclosure in any way, in an aspect of the present disclosure, which may be combined with any other aspect listed herein unless specified otherwise, a method for pandemic and connection tracking is provided. The method comprises collecting anonymous tracking data from a wireless sensor network, wherein the wireless sensor network is configured to communicate with users' devices using a wireless connection; validating the collected anonymous tracking data; estimating an exposure time between each pair of users based on the collected anonymous tracking data; and determining a probability of at least one of the users being infected based on the estimated exposure time.

In an aspect of the present disclosure, which may be combined with any other aspect listed herein unless specified otherwise, the anonymous tracking data comprises a MAC address of the user's device.

In an aspect of the present disclosure, which may be combined with any other aspect listed herein unless specified otherwise, the method further comprises determining whether any anonymous tracking data is missing; and responsive to determining that any anonymous tracking data is missing, augmenting the collected anonymous tracking data.

In an aspect of the present disclosure, which may be combined with any other aspect listed herein unless specified otherwise, validating the collected anonymous tracking data comprises comparing an average distribution of the collected anonymous tracking data with a predetermined model.

In an aspect of the present disclosure, which may be combined with any other aspect listed herein unless specified otherwise, validating the collected anonymous tracking data comprises comparing an average distribution of exposure times in the collected anonymous tracking data with an average distribution of exposure times derived using a predetermined model.

In an aspect of the present disclosure, which may be combined with any other aspect listed herein unless specified otherwise, the probability of at least one of the users being infected is determined further based on a distance level of the pair of users during the estimated exposure time.

In an aspect of the present disclosure, which may be combined with any other aspect listed herein unless specified otherwise, the distance level comprises: a high risk distance level indicating that a distance between the pair of users is lower than a first predetermined distance; a medium risk distance level indicating that the distance between the pair of users is greater than the first predetermined distance but lower than a second predetermined distance; and a low risk distance level indicating that the distance between the pair of users is greater than the second predetermined distance.

In an aspect of the present disclosure, which may be combined with any other aspect listed herein unless specified otherwise, the method further comprises detecting a security attack by: recording a distance between one of the users and other users among the users for a set period of time, calculating an average distribution of the distance between the one of the users and the other users; and measuring a Hellinger distance error based on the calculated average distribution.

The reader will appreciate the foregoing details, as well as others, upon considering the following detailed description of certain non-limiting embodiments including a method and system for pandemic and connection tracking according to the present disclosure.

### BRIEF DESCRIPTION OF THE DRAWINGS

FIG. 1 is a diagram of a system for pandemic and connection tracking according to an example embodiment of the present disclosure.

FIG. 2 is a diagram of a wireless sensor network (e.g., IoT proximity tracking network) for the system of FIG. 1 according to an example embodiment of the present disclosure.

FIG. 3 is a diagram of wireless sensors of the system of FIG. 1 installed in a public zone, collecting Wi-Fi and/or Bluetooth beacons according to an example embodiment of the present disclosure.

FIG. 4 is a diagram showing a concept of some example risk zones for the algorithm of the system of FIG. 1 according to an example embodiment of the present disclosure.

FIG. 5 is a flowchart showing iterative infection probability computation for the algorithm for the system of FIG. 1 according to an example embodiment of the present disclosure.

FIG. 6 is a flowchart showing spread factor estimation (SFE) algorithm for the system of FIG. 1 according to an example embodiment of the present disclosure.

FIG. 7 is a flowchart showing iterative Bisect algorithm for the system of FIG. 1 according to an example embodiment of the present disclosure.

FIG. 8 is a diagram showing potential security attacks.

FIG. 9 is a diagram of a system for pandemic and connection tracking according to an example embodiment of the present disclosure.

FIG. 10 is a diagram showing an example of simulated virus spread tracking for one week.

### DETAILED DESCRIPTION

The present disclosure generally relates to a method and system for pandemic and connection tracking. Several pandemics have spread all over the world in the recent human history and caused major economical and social damages. In particular, Covid-19 virus is the most recent and has caused the death of more than a million people so far around the globe. Therefore, the design of a model that can predict the persons that are most likely to be infected is a necessity to stop any pandemic.

Several works have been performed to control the pandemic, some countries made use of smart phone applications that track the movement of individuals to warn the healthy ones with the proximity of suspected infected cases. However, the implementation of such applications may be very challenging since it may require the permission to GPS or Bluetooth access for every subscribed user. In particular, a flood of COVID-19 applications may be tracking people/users today and certain number of them might be pervasive and invasive. Although these applications may be helpful in tracing the virus spread, with such huge amount of private data, it is inevitable for some of these contact-tracing apps to have data leaks which might create a dramatic privacy problem. Also, these apps might serve as an excuse for abuse and disinformation.

One of the major challenges such applications are facing is the social stigma. In particular, many people may avoid using these applications for two main reasons. The first worry raises from the lack of transparency of some of these applications and the potential malicious use of the collected private data. The second worry is that for many persons, shame can be worse than the infection itself. In particular, such behavior might raise from worries of discrimination, blame, ignorance, fear and anxiety. In particular, medical shaming often causes blatant disregard or disgust for the patient itself. Furthermore, there have been no application in the past that gives the users the probability of being infected (or in connection with some person) even when never getting close to someone with a confirmed infection.

In the present disclosure, an internet of things (IoT) sensing network may be designed to anonymously track the movement of individuals in crowded zones using low cost WiFi and Bluetooth devices. This may be done by collecting both WiFi and Bluetooth beacons from the mobile phones to triangulate and estimate the locations inside buildings without violating the user's privacy. Some algorithm may be presented to compute the expected time of exposure between users to replace missing measurements. Furthermore, a virus spread algorithm model as well as iterative spread tracking algorithms are also proposed to make use of this map to predict the probability of individuals being infected even with limited data.

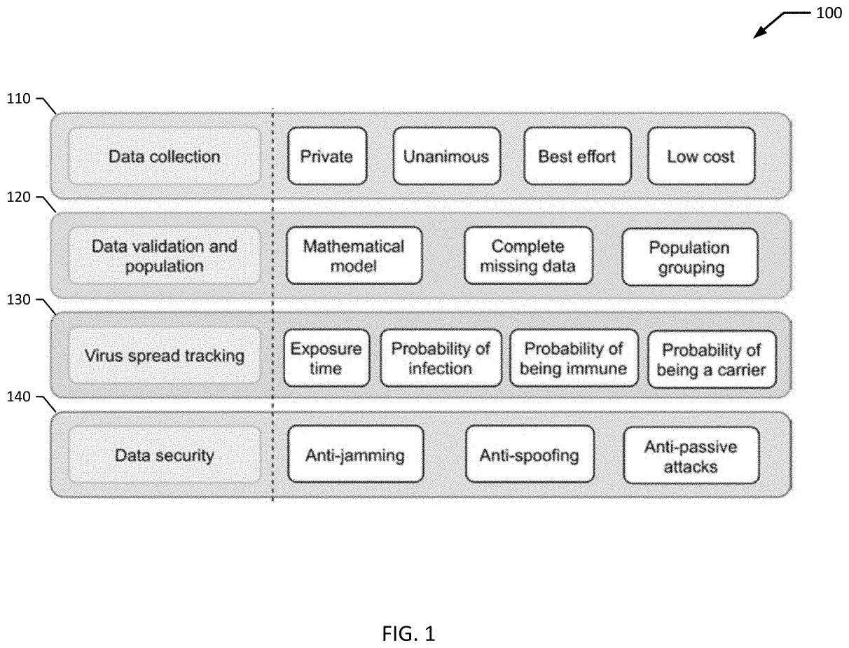

According to an embodiment of the present disclosure, a system 100 for pandemic and connection tracking may be provided. The system 100 may include four layers as shown in FIG. 1. The data collection layer 110 may allow the anonymous collection of the location and proximity data using low cost IoT sensing network. The data validation and augmentation layer 120 may augment data, when the tracking data is missing, using a specially designed algorithm/mathematical model. This might be beneficial if a limited number of sensors are available and the users cannot always be tracked. Also, by comparing the distribution of the average collected data with the algorithm/mathematical model, the data can be validated. In the virus spread tracking layer 130, by making use of the collected and computed data, the time of exposure between each pair of individuals may be computed. Also, by defining three exposure levels (high, medium and low), and by fitting the system configuration parameters, an algorithm/mathematical model may be used to compute the probability of each person being infected, not infected or immune. In the data security layer 140, using the algorithm/mathematical model, some metrics may be created to detect several potential attacks, such as jamming, spoofing and passive attacks. These four layers may operate together to ensure low-cost, anonymous, efficient, transparent, secure and accurate estimation of infection/contact tracking.

In some examples, the system 100 may be based on the anonymous continuous monitoring of users in public crowded zones using IoT wireless sensor networks. The tracking may be performed to ensure the individual's health safety while preserving their privacy and without any required permission form users. Since most of the smartphone users keep the Bluetooth and/or WiFi active even when going in public areas, the smartphones of the users may regularly send wireless beacons trying to identify potential networks. Therefore, by installing low cost wireless devices (e.g., ESP32) at locations that are most likely to be visited by a large number of people (supermarkets, train stations, bus stops, etc.) both WiFi and Bluetooth beacons can be automatically collected. These beacons may include the unique MAC addresses of the users that can be used to build an anonymous movement tracking data.

The system 100 may be able to cover a massive amount of public zones on large widespread areas by using the wireless sensor networks (WSNs) that may incorporate low-cost, energy efficient and robust devices that may be setup in various/targeted/optimized locations. By using small-sized and low-cost sensors with wireless data transfer capabilities (e.g., ESP32), a wide-range of fields can be covered by the WSNs.

In some examples, the collected data may be kept anonymous and/or linked only to the MAC addresses of the devices of the users. Contrary to personal information, the MAC address of user devices may not have sensitive information as long as it is not linked to the person identity. In some examples, in the system 100, even if a user gets an expected high infection rate, no one can know the identity of this person. In some examples, the system 100 may allow an individual with a high probability of infection that has entered a public zone to send an alert message to other users' devices using his MAC address, or sending an alert to the authorities that can come, identify him using triangulation techniques and perform necessary isolation and follow-up procedure.

In some examples, a (mobile) application (in the users' devices) can be linked to the system 100 without requesting any personal information from the users. In particular, this application can be used to report to the user his probability of being infected each day without knowing his identity.

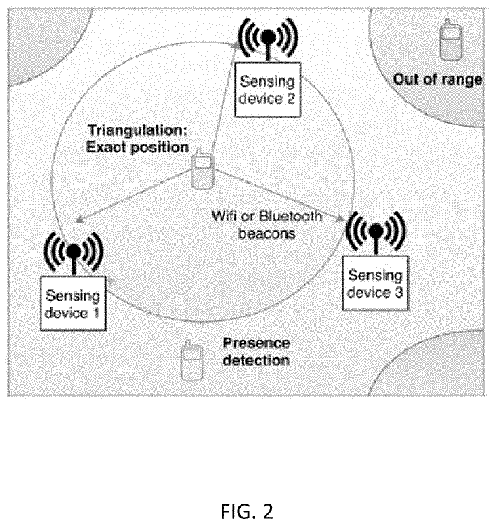

In some examples, for accurate tracking, each set of sensors may be placed in very specific locations according to the zones architectures and characteristics. As shown in FIG. 2, these locations may be optimized in order to detect most of the social contacts with as less devices and as less cost as possible. Therefore, this network may focus more on the crowded parts of zones, such as cashiers, where the system 100 can obtain accurate readings of the exact positions, for example, using wireless beacons triangulation (blue circle areas in the middle of FIG. 2). For the non-crowded parts of the building, the system 100 may just detect the presence of users using less sensors (e.g., a single sensing device (orange areas outside of the blue circle in FIG. 2). Even when adopting such strategy in the design of the IoT sensing network, there could be some dead-areas where none of the installed sensing devices will have a reach (red areas in the corners in FIG. 2). Therefore, the system 100 may utilize an investigative prediction algorithm that can replace these readings.

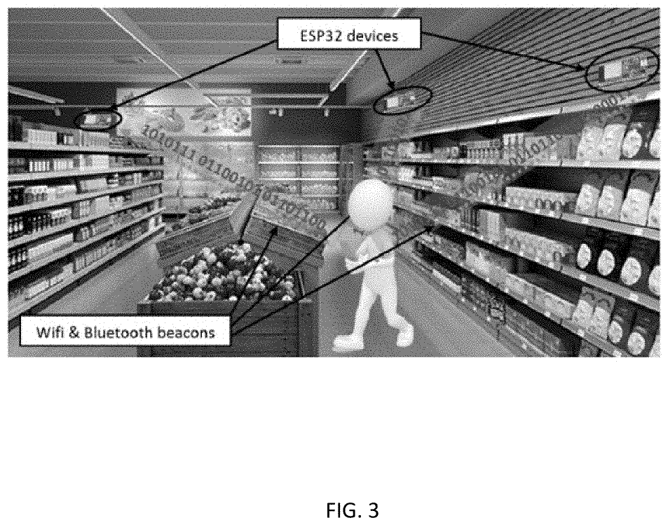

In some examples, for some installed sensors, the grid power line may be close and it may be more efficient to power them directly from the grid (see FIG. 3). However, some other sensors may be placed in locations with no direct access to the grid or may require further installations to link them to the grid which could slow down the setup of the IoT tracking system. Thus, in some examples, the sensor devices may be configured to increase the battery life by using energy efficient technique and by regularly recharging the batteries. In some cases, since the crowded zones may be indoor, there may be limited or no access to traditional renewable energy sources, such as sunlight. In those cases, some of the sensor devices may be powered using energy harvesting techniques.

In some examples, the system 100 may use derived mathematical expressions/algorithm of an exposure time between each pair of users. This can be used to verify the validity of the reported data by comparing the average distribution of the collected and derived exposure times. This can also help the detection of various types of attacks that aim to falsify the reported data. In some cases, it might be difficult to obtain the exact position of each person in all of the target (e.g., public) zones at all times. Therefore, in some cases, the system 100 may approximate the exposure time between two users just based on the fact that they were at the same zone. This exposure time can be approximated based (solely) on the probability of the users going into a specific public zone.

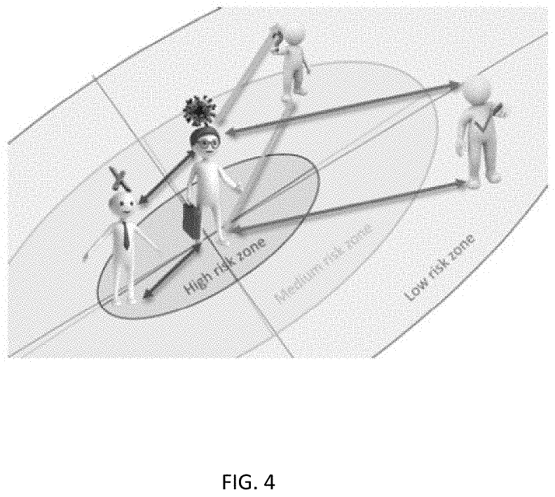

This algorithm model may be based on the definition of three risk levels. High risk level (HRL) may indicate that the distance between the two users is smaller than a first predetermined distance (e.g., r₁=1 m), as shown in the core central red region in FIG. 4. Medium risk level (MRL) may indicate that the distance between the two users is between the first predetermined distance (e.g., r₁=1 m) and a second predetermined distance (e.g., r₂=2 m). Low risk level (LRL) may indicate that the distance between the two users is greater than the second predetermined distance, for example, between the second predetermined distance (e.g., r₂=2 m) and a third predetermined distance (e.g., r₃=7 m).

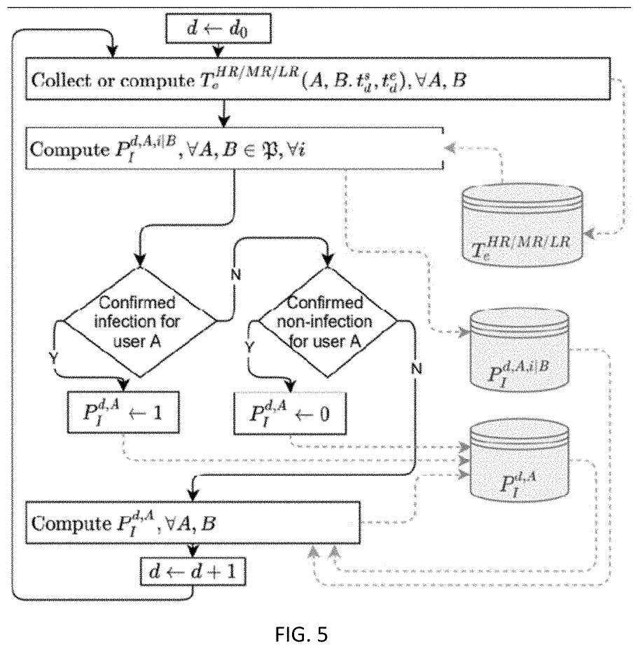

In some examples, the system 100 may obtain the probability of getting the infection at day d by user A denoted as P_{I}^{d,A} iteratively based on all the probabilities of getting the infection from day d₀ till day d−δ₁ for all the users in the investigated population P_{OP} (see FIG. 5). This relation may be made possible by exploiting the high risk, medium risk and low risk exposure times between each pair of users (A, B) denoted by Tₑ^{HR\MR\LR} (AA, BB, start time, end time). In particular, the computation of time of exposure between each pair of users in each target (public) zone and in each risk zone may be first performed assuming uniformly distributed users in the zones. These values may then be used to design and estimate a virus-spread/connection map. If a set of users are confirmed to get the infection at day d*, the system 100 may be able to update the probability of any other user in the community to be infected.

Since the user may stay infected for δ₂ days, the probability of being infected P_{B,I}^{d,A}: may be equivalent to the probability of not being immune and getting the infection at least once from day d−δ₂ till day d. To transform the relation between the different investigated probabilities into a virus spread map, the system 100 may utilize the iterative algorithm in FIG. 5. The inputs of this algorithm may be the set of confirmed infections at each day d denoted by C(d), the set of confirmed non infected persons at each day d denoted by N(d) and the movement tracking map denoted M of all the users (or their distances distributions) that allow the computation of the times of exposure. The outputs of the algorithm may be the probability of getting the infection, being infected, being immune and being contagious for each user A and for each day d during the investigated period D.

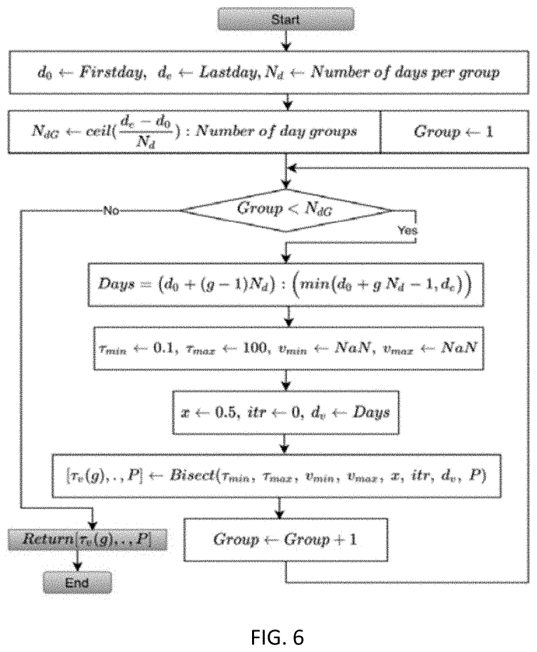

In some examples, to estimate the adequate spread factor vector τᵥ, the system 100 may use the Spread Factor Estimation (SFE) algorithm shown in FIG. 6. The SFE algorithm may start by defining the first and last investigated days d₀ and dₑ as well as the number of day groups N_{dG}. For each group Group from 1 to N_{dG}, the set of investigated days may be defined as Days.

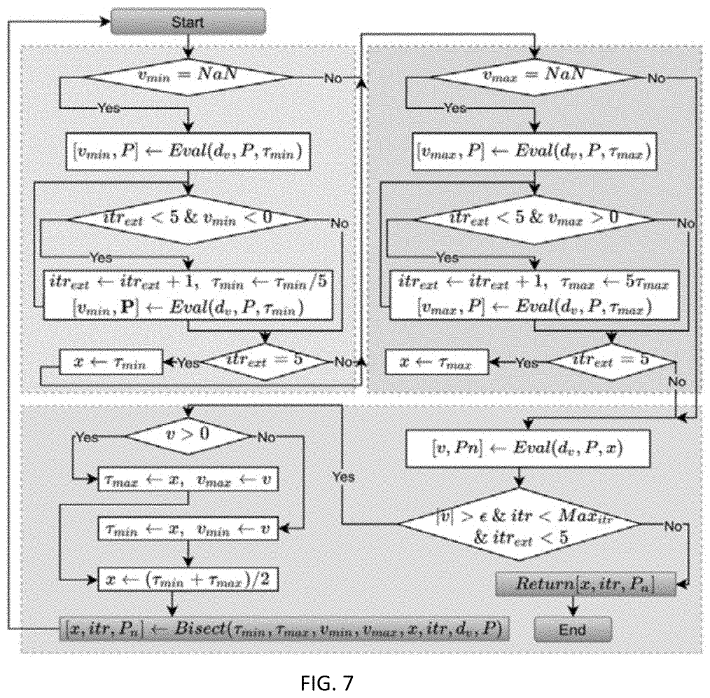

For each set Days, the system 100 may use the bisection (Bisect) algorithm in FIG. 7 to estimate τᵥ (Group) and the set of probabilities P, where P denotes the probabilities of infection, being infected, being immune and being contagious for all the investigated period and for all the investigated population. In some examples, to predict the infections in a group of days Group, all the probabilities since d₀ may be used. However, only the probabilities in the days that belong to Group may change in P. Therefore, P may be created iteratively group by group according to the Bisect algorithm. The Bisect algorithm may be called with the minimum and maximum potential spread factors and their corresponding errors may be set to NaN to make sure that the algorithm computes their errors in the beginning.

The Bisect algorithm may use the function [v, P]←Eval (dᵥ, P, τ) that may calculate all the probabilities P given the initial P and the spread factor τ during the set of days Days. The expected number of active cases at the end of the investigated group of days N_{A} may be computed as the expectation of the probability of being infected over all the population. Finally, the difference between the reported number of active cases and the computed one may be returned as v. Eval may make use of P for all the days since d₀ but update it only for the days in Days.

For each group of days Days, a lookup for the adequate τ may be done using the Bisect algorithm in FIG. 7 from τₘᵢₙ to τₘₐₓ. Since the expected number of active cases is a decreasing function of τ, the error vₘᵢₙ when using τₘᵢₙ may be positive (Expected number higher than the reported one). Similarly, the error vₘₐₓ when using τₘₐₓ may be negative. To make sure that the interval [τₘᵢₙ, τₘₐₓ] contains the desired τ, the system 100 (e.g., using the Bisect algorithm) may start by computing vₘᵢₙ and vₘₐₓ and making sure that vₘᵢₙ≤0 and vₘₐₓ≥0. Otherwise, τₘᵢₙ may be reduced to τₘᵢₙ/5 and/or 5τₘₐₓ up to 5 times (itrₑₓₜ<10). In case the extension of τₘₐₓ or τₘᵢₙ 10 times does not make the system satisfy the initial constraints, then τ that had the smallest error may be reported. This may help avoid rare divergence cases that may occur due to some extreme conditions or sometimes because of a large number of deaths that is not taken into consideration in this model. Once the initial conditions are satisfied, the error v may be computed for the potential τ denoted by x. Then, at each iteration, x may be set to

$$\frac{x + {τ}_{min}}{2}$$

if v>0 and to

$$\frac{x + {τ}_{max}}{2}$$

if v<0 till reaching either the maximum number of iterations Maxᵢₜᵣ or the minimum absolute error (|v|≤∈). Once one of these conditions is reached the Bisect algorithm may return P and τ=x.

Some users may not prefer to share their private location data. Therefore, if they are aware of the presence of such tracking system in a public zone, they might switch off their Bluetooth and/or WiFi. To avoid this, one possible solution could be to make sure at the entrance that the WiFi and/or Bluetooth of the mobile phone of the user is switched ON before entering, for example, by checking the status in mobile applications. Even in such scenarios, the individuals might switch off the Bluetooth and/or WiFi once entering into the zone to falsify the result. Also, there could be an attempt to make spoofing attacks where the users send beacons with falsified MAC address. In some examples, such behavior of a user A can be detected by recording the distance between A and all the other users in the target (e.g., public) zone for the visited duration. This data may be then used to compute the average distribution of the distance between A and all the other users denoted F_{dA,B}^{m}. Consequently, the Hellinger distance between the measured and derived A,B distributions can be an efficient tool to identify the falsified data.

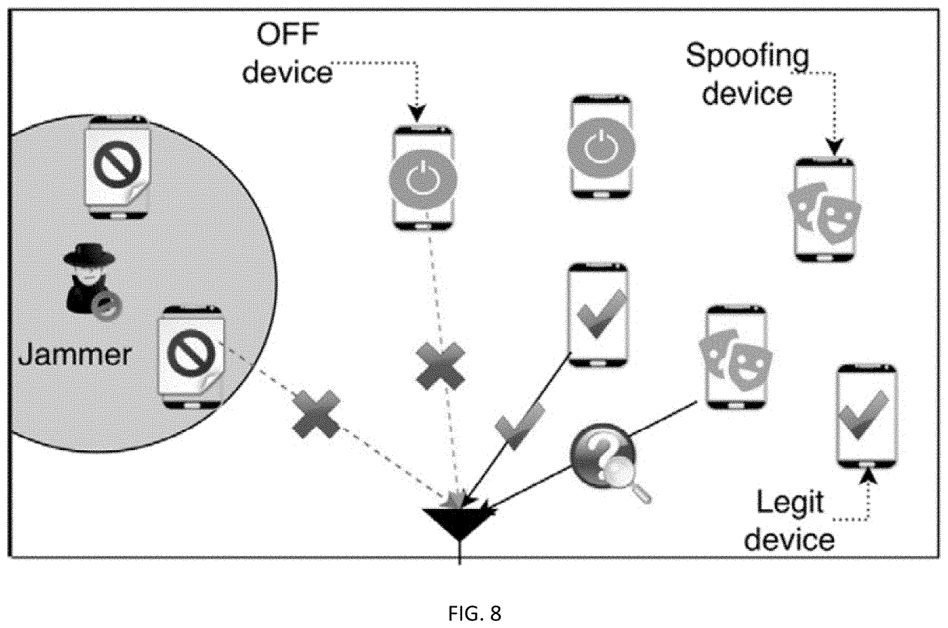

In some examples, the system 100 may use the Hellinger distance to measure the difference between the measured and the theoretical distribution. In some examples, the system 100 may use the Hellinger distance threshold in detecting malicious users that try to falsify the data by jamming the devices that collect the beacons. In particular, the jamming may create non-reachable areas which would change the measured public zone dimensions and therefore the distribution of the distances which would raise the Hellinger distance error. Therefore, by measuring the Hellinger distance error, the system 100 may become robust against several types of attacks. The system 100 may avoid these attacks by setting an error threshold which may detect jamming, spoofing or passive attacks, and the system 100 may report and correct the attacks (See FIG. 8). Additional descriptions of some algorithms that can be used by the system 100 can be found in “A Novel Pandemic Tracking Map: From Theory to Implementation,” Gouissem et al., IEEE Access (Volume: 9), 51106-51120 (31 Mar. 2021), the disclosure of which is hereby incorporated by reference in its entirety.

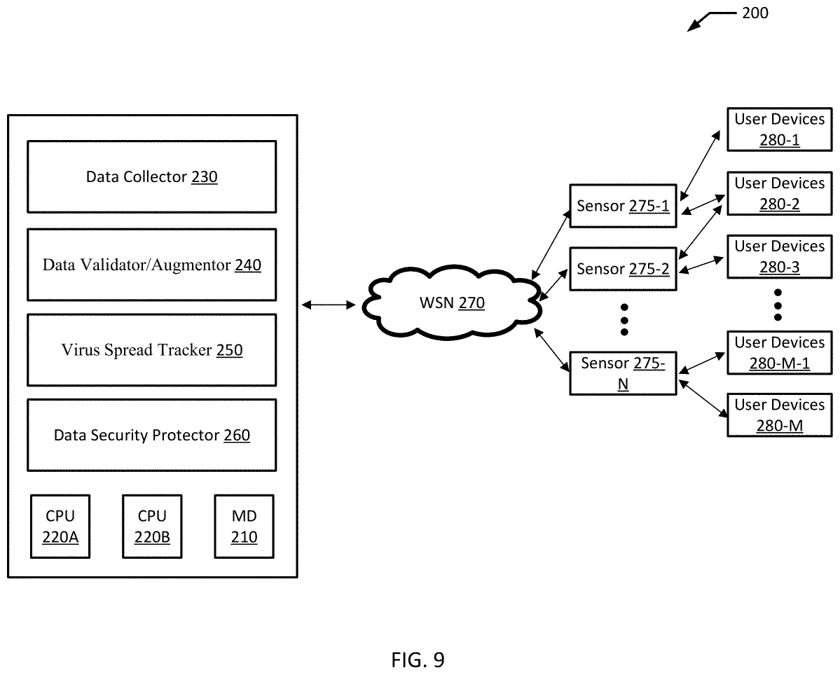

FIG. 9 illustrates a system 200 for pandemic and connection tracking according to an example embodiment of the present disclosure. As shown in FIG. 9, the system 200 may include a memory 210 and one or more processors 220A-220B in communication with the memory 210. The system 200 may further include a data collector 230 executing on the one or more processors 220A-220B. The data collector 230 may collect anonymous tracking data from a wireless sensor network 270. The wireless sensor network 270 may communicate with users' devices 280-1-280-M using a wireless connection.

The system 200 may further include a data validator/augmentor 240 executing on the one or more processors 220A-220B. The data validator/augmentor 240 may validate the collected anonymous tracking data.

The system 200 may also include a virus spread tracker 250 executing on the one or more processors 220A-220B. The virus spread tracker 250 may estimate an exposure time between each pair of users based on the collected anonymous tracking data and determine a probability of at least one of the users being infected based on the estimated exposure time.

In some examples, the anonymous tracking data may include beacons transmitted from the user's devices. In some examples, the anonymous tracking data may include a MAC address of the user's device 280-1-280-M. in some examples, the MAC address may be included in the beacons transmitted from the user's devices.

In some examples, the data validator/augmentor 240 may determine whether any anonymous tracking data is missing. Responsive to determining that any anonymous tracking data is missing, the data validator/augmentor 240 may augment the collected anonymous tracking data. In some examples, the data validator/augmentor 240 may validate the collected anonymous tracking data by comparing an average distribution of the collected anonymous tracking data with a predetermined (algorithm) model, as discussed above. In some examples, the data validator/augmentor 240 may validate the collected anonymous tracking data by comparing an average distribution of exposure times in the collected anonymous tracking data with an average distribution of exposure times derived using a predetermined (algorithm) model.

In some examples, the system 200 may further include a plurality of wireless sensors 275-1-275-N. In some examples, the plurality of wireless sensors 275-1-275-N may be part of the wireless sensor network 270. The plurality of wireless sensors 275-1-275-N may detect the anonymous tracking data and transmit the anonymous tracking data to the data collector 230. In some examples, the plurality of wireless sensors 275-1-275-N may include a set of sensors surrounding a location of interest for wireless triangulation. For example, the location of interest may be a crowded part of the target (public) zone (e.g., near the cashier in a store).

In some examples, the virus spread tracker 250 may determine a probability of at least one of the users being infected further based on a distance level of the pair of users during the estimated exposure time. The distance level may include a high risk distance level, a medium risk distance level, and a low risk distance level. The high risk distance level may indicate that a distance between the pair of users is lower than a first predetermined distance (e.g., 1 m). The medium risk distance level may indicate that the distance between the pair of users is greater than the first predetermined distance but lower than a second predetermined distance (e.g., 2 m). The low risk distance level may indicate that the distance between the pair of users is greater than the second predetermined distance.

In some examples, the system may further include a data security protector 260 executing on the one or more processors 220A-220B. The data security protector 260 may detect a security attack. For example, the data security protector 260 may detect a security attack by recording a distance between one of the users and other users among the users for a set period of time, calculating an average distribution of the distance between the one of the users and the other users; and measuring a Hellinger distance error based on the calculated average distribution.

Aspects of the present disclosure may advantageously provide a design of a transparent, anonymous and private tracking system and enable an accurate real-time infection/contact tracking. The system and method according to the present disclosure may insure the privacy and security of the sensitive private data. Also, unlike the conventional system, aspects of the present disclosure may not require a special permission or application installation by the user.

Aspects of the present disclosure may also provide the users with information about the probability of infection and the probability of being immune instead of just classifying them into “infected,” “not infected,” and/or “potentially infected”. Aspects of the present disclosure can be applied to other contact tracing applications.

Although it might be challenging to install sensors to cover all the public zones, aspects of the present disclosure may resolve this issue by: installing low-cost, low-energy sensors which allow the coverage of large areas with limited budgets; installing sensors (only) in the most crowded zones where most of the infections occur; make use of the data augmentation techniques to estimate the missing data in the non-covered zones; and/or installing (only) one sensor in zones that are less crowded (the presence and absence of the users can be confirmed by the collected signals, while the exact locations can be approximated using the system and the above discussed algorithm).

The system was simulated and analyzed using extensive computer Monte-Carlo simulations. In particular, using realistic community configurations and approximations, and by using the actual number of reported Covid-19 active cases in few countries, the efficiency of the system in fitting into the real reported data has been confirmed and the results of the simulations confirm the accuracy of the system in tracking the virus spread.

The reproduction number of an infectious disease denoted RO is a parameter widely used to evaluate the speed of spread of viruses. It may refer to the number of people to which an infected individual can directly transmit the disease during the infection period. Its values can reach up 18 for very contagious diseases such as measles, and it is around 3 for COVID-19. In particular, the proposed model defines a spread factor parameter τ that reflects how much time a user has to stay in a contagious zone to get infected. This parameter may depend on lots of variables, such as the type of the virus, the level of social distancing, how many people are using facial masks, how frequently people are disinfecting their hands, and so on. Therefore, special techniques may be provided in the present disclosure to estimate τ using realistic reported data which may make the present disclosure, with a few parameter tuning, very suitable for several pandemic spread tracking applications related to H1N1, SARA-CoV, Tuberculosis, Covid-19 and any potential novel pandemics.

Furthermore, by forcing the immunity probability to 0, and setting the contagious duration to infinity, this tracking method according to the present disclosure may be used for several non-health related applications such as tracing criminals and their potential contacts.

The present disclosure can be used, for example, by governmental authorities and healthcare providers. First, healthcare providers can make use of this model to track the spread of Covid-19 and any type of respiratory contagious disease, such as H1N1, SARS-Cov, Tuberculosis as well as any other potential pandemic. In particular, the method according to the present disclosure may be able to track the virus spread accurately and efficiently which provides healthcare providers with the necessary information to take the adequate decisions in time to stop and control the pandemic spread.

Also, governmental authorities can make use of the system and method according to the present disclosure, for example, by applying it any contact tracing application. In particular, concerning the health related and pandemic tracking applications, hospitals, ministry of Health, schools as well as the ministry of transportation can be interested in using the health related and pandemic tracking applications. Aspects of the present disclosure may allow the authorities to control and track the spread of any contagious disease in public areas, such as schools, supermarkets and public transportations, for example, by allowing only users with low probability of infections to enter such areas.

For example, once any patient has been confirmed to be infected at day d, the doctors can approximate that the patient was infected at date d₀. Then, the MAC address can be extracted from the patient's smart-phone and the map may be used to estimate the probability of other people to be infected because of the patient in the previous days. However, since the data is anonymous, it is not straightforward to know the identity of the people highly probable to be infected. Therefore, the installed devices may be programmed to raise an alert when the probability of a user to be infected with the disease is equal to or greater than a predetermined threshold. Then, the authorities can identify the infected individual and take him for testing and further medical follow-up. These devices can also send the user a warning message using his MAC address telling him to seek medical advice or to stay at home.

Therefore, aspects of the present disclosure may help track the virus spread and faster control of the pandemic. Furthermore, with the scheduled public facilities reopening, and with the discovery of new variants of Covid-19 virus, it is likely that there would be a spike in the spread of the disease again (which already happened in several countries). It is also expected by the World Health Organization (WHO) that there could be multiple Covid-19 virus spread waves. Aspects of the present disclosure may help the society address this situation, for example, by quickly tracking the spread of the disease.

Because aspects of the present disclosure do not require any special permission or mobile applications, it may also help control crimes, gang activities, or terrorist behaviors. In particular, this can be done by tracking criminals and computing their historical contact maps and the probability of interaction between them. Aspects of the present disclosure may quickly create a potential list of people in contact with a detained criminal with the probability as well as the duration of contact. In some examples, for criminal tracking purposes, the identity of the criminal connections may be revealed by the corresponding authorities that can be alerted once a sensing device detects their presence in a public area. Similarly, aspects of the present disclosure can also be used to track the spread of radioactive and chemical poisoning.

### Example

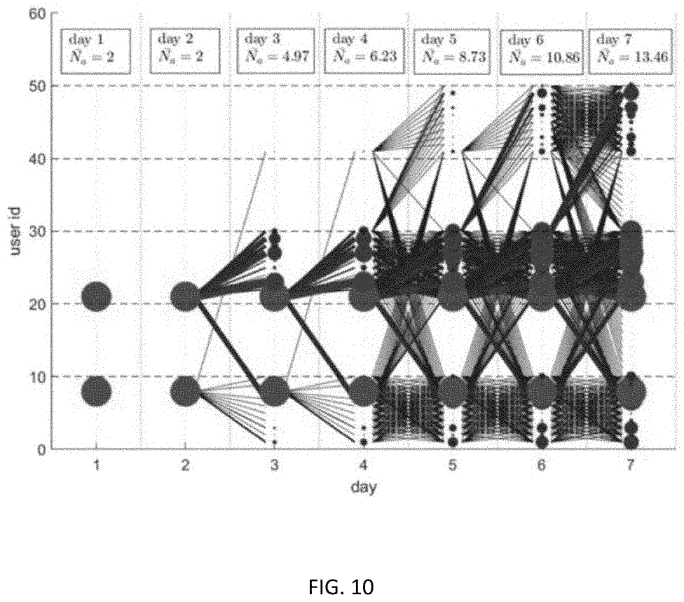

To visualize the virus spread, a small community is simulated for one week, as shown in FIG. 10. This community is assumed to be composed of 5 groups with 10 persons in each. One out of the 10 persons in each group is assumed to also move in another group simultaneously. Each group of users is assumed to randomly visit five public zones of size 50 m×50 m.

At day 1, only 2 out of the 50 persons are assumed to get confirmed infections; user 8 in group 1 and user 21 in group 3. Since user 8 and 21 became infected at day 1, they will not become contagious before day three when they start spreading the virus over the persons they came close to. In particular, even-though these two infected individuals came in contact with several users in their groups, the proposed model did not predict whether any other user in any group would have any chance of infection till day 3 when they became contagious. At day 3, user 21 spread the virus over several members of his group. As shown in FIG. 10, not all the members in group 3 had the same probability of being infected since they are modeled to have different average times spent in public zones and they might go to different zones. User 21 is assumed to go to both the zones of group 1 and 5 which caused the infection of user 39. Again, user 21 spread the disease in his group only 2 days after his infection (starting from day 5). Furthermore, the infections spread more and more over time. In particular, even if a member of a group does not catch the virus in the previous days from the confirmed infected persons, he/she may start to get some chances of infection due to other new non-confirmed but probable cases.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.10% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

When the position relation between two parts is described using the terms such as “on,” “above,” “below,” “under,” and “next,” one or more parts may be positioned between the two parts unless the terms are used with the term “immediately” or “directly.” Similarly, as used herein, the terms “attachable,” “attached,” “connectable,” “connected,” or any similar terms may include directly or indirectly attachable, directly or indirectly attached, directly or indirectly connectable, and directly or indirectly connected.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “at least one of X or Y” or “at least one of X and Y” should be interpreted as X, or Y, or X and Y.

It will be appreciated that all of the disclosed methods and procedures described herein can be implemented using one or more computer programs or components. These components may be provided as a series of computer instructions on any conventional computer readable medium or machine readable medium, including volatile or non-volatile memory, such as RAM, ROM, flash memory, magnetic or optical disks, optical memory, or other storage media. The instructions may be provided as software or firmware, and/or may be implemented in whole or in part in hardware components such as ASICs, FPGAs, DSPs or any other similar devices. The instructions may be configured to be executed by one or more processors, which when executing the series of computer instructions, performs or facilitates the performance of all or part of the disclosed methods and procedures.

The example embodiments may be embodied in the form of computer-implemented processes and apparatuses for practicing those processes. An embodiment may also be embodied in the form of a computer program code containing instructions embodied in tangible media, such as floppy diskettes, CD-ROMs, DVD-ROMs, hard drives, or any other computer readable non-transitory storage medium, wherein, when the computer program code is loaded into and executed by a computer, the computer becomes an apparatus for carrying out the method. An embodiment may also be embodied in the form of computer program code, for example, whether stored in a storage medium, loaded into and/or executed by a computer, or transmitted over some transmission medium, such as over electrical wiring or cabling, through fiber optics, or via electromagnetic radiation, wherein when the computer program code is loaded into and executed by a computer, the computer becomes an apparatus for carrying out the method. When implemented on a general-purpose microprocessor, the computer program code segments configure the microprocessor to create specific logic circuits.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims. Moreover, consistent with current U.S. law, it should be appreciated that 35 U.S.C. 112(f) or pre-AIA 35 U.S.C. 112, paragraph 6 is not intended to be invoked unless the terms “means” or “step” are explicitly recited in the claims. Accordingly, the claims are not meant to be limited to the corresponding structure, material, or actions described in the specification or equivalents thereof.

The invention is claimed as follows:

1. A system for pandemic and connection tracking, the system comprising:
a memory;
one or more processors in communication with the memory;
a data collector executing on the one or more processors, wherein the data collector is configured to collect anonymous tracking data from a wireless sensor network, wherein the wireless sensor network is configured to communicate with users' devices using a wireless connection;
a data validator/augmentor executing on the one or more processors, wherein the data validator/augmentor is configured to validate the collected anonymous tracking data; and
a virus spread tracker executing on the one or more processors, wherein the virus spread tracker is configured to:
estimate an exposure time between each pair of users based on the collected anonymous tracking data; and
determine a probability of at least one of the users being infected based on the estimated exposure time.

2. The system according to claim 1, wherein the anonymous tracking data comprises beacons transmitted from the user's devices.

3. The system according to claim 1, wherein the anonymous tracking data comprises a MAC address of the user's device.

4. The system according to claim 1, wherein the data validator/augmentor is further configured to:
determine whether any anonymous tracking data is missing; and
responsive to determining that any anonymous tracking data is missing, augment the collected anonymous tracking data.

5. The system according to claim 1, wherein validating the collected anonymous tracking data comprises comparing an average distribution of the collected anonymous tracking data with a predetermined model.

6. The system according to claim 1, wherein validating the collected anonymous tracking data comprises comparing an average distribution of exposure times in the collected anonymous tracking data with an average distribution of exposure times derived using a predetermined model.

7. The system according to claim 1, further comprising a plurality of wireless sensors in the wireless sensor network configured to detect the anonymous tracking data and transmit the anonymous tracking data to the data collector.

8. The system according to claim 6, wherein the plurality of wireless sensors comprise a set of sensors surrounding a location of interest for wireless triangulation.

9. The system according to claim 1, wherein the probability of at least one of the users being infected is determined further based on a distance level of the pair of users during the estimated exposure time.

10. The system according to claim 9, wherein the distance level comprises:
a high risk distance level indicating that a distance between the pair of users is lower than a first predetermined distance;
a medium risk distance level indicating that the distance between the pair of users is greater than the first predetermined distance but lower than a second predetermined distance;
a low risk distance level indicating that the distance between the pair of users is greater than the second predetermined distance.

11. The system according to claim 1, further comprising a data security protector executing on the one or more processors, wherein the data security protector is configured to detect a security attack.

12. The system according to claim 11, wherein detecting the security attack comprises:
recording a distance between one of the users and other users among the users for a set period of time,
calculating an average distribution of the distance between the one of the users and the other users; and
measuring a Hellinger distance error based on the calculated average distribution.

13. A method for pandemic and connection tracking, the method comprising:
collecting anonymous tracking data from a wireless sensor network, wherein the wireless sensor network is configured to communicate with users' devices using a wireless connection;
validating the collected anonymous tracking data;
estimating an exposure time between each pair of users based on the collected anonymous tracking data; and
determining a probability of at least one of the users being infected based on the estimated exposure time.

14. The method according to claim 13, wherein the anonymous tracking data comprises a MAC address of the user's device.

15. The method according to claim 13, further comprising:
determining whether any anonymous tracking data is missing; and
responsive to determining that any anonymous tracking data is missing, augmenting the collected anonymous tracking data.

16. The method according to claim 13, wherein validating the collected anonymous tracking data comprises comparing an average distribution of the collected anonymous tracking data with a predetermined model.

17. The method according to claim 13, wherein validating the collected anonymous tracking data comprises comparing an average distribution of exposure times in the collected anonymous tracking data with an average distribution of exposure times derived using a predetermined model.

18. The method according to claim 13, wherein the probability of at least one of the users being infected is determined further based on a distance level of the pair of users during the estimated exposure time.

19. The method according to claim 18, wherein the distance level comprises:
a high risk distance level indicating that a distance between the pair of users is lower than a first predetermined distance;
a medium risk distance level indicating that the distance between the pair of users is greater than the first predetermined distance but lower than a second predetermined distance;
a low risk distance level indicating that the distance between the pair of users is greater than the second predetermined distance.

20. The method according to claim 13, further comprising detecting a security attack by:
recording a distance between one of the users and other users among the users for a set period of time,
calculating an average distribution of the distance between the one of the users and the other users; and
measuring a Hellinger distance error based on the calculated average distribution.
